(19) United States
(12) Patent Application Publication
(10) Pub. No.: US 2014/0079757 A1
(43) Pub. Date: Mar. 20, 2014

(54) ORAL DRUG DELIVERY FORMULATIONS

(71) Applicants: Michael J. Hudson, Great Missenden (GB); Alan Palmer, Maidenhead (GB)

(72) Inventors: Michael J. Hudson, Great Missenden (GB); Alan Palmer, Maidenhead (GB)

(21) Appl. No.: 13/622,700

(22) Filed: Sep. 19, 2012

(51) Int. Cl. A61K 9/70

(52) U.S. Cl. CPC A61K 9/7007
USPC 424/443; 514/249; 514/48; 514/52; 424/484

(57) ABSTRACT

The invention generally relates to the preparation and use of consumable products for delivering a bioactive agent to an oral cavity of a mammal. In certain embodiments, the invention provides a consumable product that releases a bioactive agent within an oral cavity of a mammal, the product including a polymer matrix and one or more bioactive agents, in which the matrix is of a heterogeneous thickness.

### FIELD OF THE INVENTION

The invention generally relates to the preparation and use of consumable products for delivering a bioactive agent to an oral cavity of a mammal. In certain embodiments, the invention provides a consumable product that releases a bioactive agent within an oral cavity of a mammal, the product including a polymer matrix and one or more bioactive agents, in which the matrix is of a heterogeneous thickness.

### BACKGROUND

An important consideration for pharmaceutical companies is the route of administration of any drug. Generally, the oral route is the preferred route due to the highest acceptability by patients, and about 60% of all medicine dosage forms are available as oral solid tablets or capsules. However, oral tablets and capsules can be difficult to ingest for young children, the elderly, or patients with swallowing disorders, e.g., dysphagia or dyanophagia, stroke, damaged salivary glands or xerostomia (dry mouth). Once swallowed, the contained drug is released from the matrix, absorbed by the digestive system and enters the hepatic portal system, where it is carried through the portal vein into the liver before it circulates to the rest of the body. The liver thus metabolizes many drugs prior to their exposure to the rest of the circulatory system. Such ‘first-pass’ metabolism affects the concentration-time profile of the drug in blood, which influences the time of onset of therapeutic action, the duration of interaction with the therapeutic target or exposure to the barrier between the central nervous system and the bloodstream (the blood-CNS barrier). As a result, tablet or capsule formulations often require higher doses of active drug and generally have a delayed onset of action compared to the delivery of the native drug compound directly into the blood stream. Furthermore, certain drugs are not suitable for oral administration because they are poorly absorbed, even after they are dispersed in the stomach, due to low solubility and slow dissolution in the gastric fluids or modification of the active drug. It is also commonplace that first pass metabolism may occur to such an extent that only a small amount of active drug emerges from the liver to reach the rest of the circulatory system.

To address those limitations, orally-dissolving films have been developed. Orally-dissolving films generally include a thin strip of a solid dosage form, often freeze-dried and sugar-based, incorporating a pharmacologically active drug. The films are very thin (e.g., less than 0.5 mm in thickness) and are designed to rapidly disintegrate in the oral cavity upon exposure to saliva in order to release the active drug from the film. Orally-dissolving films are especially beneficial for pediatric, geriatric and patients where proper and complete swallowing can be difficult. Additionally, the ability of orally-dissolving films to dissolve rapidly without the need for water provides an alternative to patients with swallowing difficulties.

However, the rapid dissolution and bolus release of the active drug into a patient's system is also a problem with orally-dissolving films. Since the active drug is released in an uncontrolled manner as a single bolus, it is important to limit the amount of drug that is loaded into the film, ensuring that a patient does not receive a one-time excessive dose of the active drug. Additionally, the minimal thickness of the film limits the amount of drug that can be loaded into the film.

### SUMMARY

The invention provides compositions that allow for the controlled release of a bioactive agent within an oral cavity of a mammal. Aspects of the invention are accomplished by providing a product that includes a polymer matrix that has a heterogeneous thickness. The invention recognizes that surface area and thickness of the matrix affect the rate at which the bioactive agent is released from the matrix. By varying the surface area and thickness in a single matrix, the agent is released from the product at different rates, thereby allowing for controlled release of the bioactive agent over time, rather than a single bolus release of a bioactive agent as might be experienced from a film preparation. By being able to provide for controlled release of a bioactive agent from the matrix, compositions of the invention are safer and are also able to accommodate higher drug loading that orally-dissolving films which are typically of a single uniform thickness.

In particular aspects, compositions of the invention are formulated as products that include a polymer matrix having one or more areas of reduced thickness, such as perforations in the film (i.e., holes through the film). The perforations increase the surface area of the product and reduce the diffusion distance for release of active drug ingredients, allowing for an initial rapid release of agent from the matrix, while the bioactive agent in thicker sections of the matrix is released at a slower rate. In this manner, compositions of the invention provide a predictable initial bolus release followed by controlled and sustained release over an extended time frame compared to normal orally-dissolving films., e.g., selectable over seconds or tens of minutes.

Generally, compositions of the invention include a polymer matrix and one or more bioactive agents. The type of polymer chosen affects the dissolution rate of the matrix, and thus also affects the rate of release of the agent from the matrix. In certain embodiments, the matrix is made of a single polymer. In other embodiments, the polymer matrix includes a plurality of different polymers, optionally having different dissolution rates. Generally, when using multiple polymers, the different polymers are arranged as different layers, which allows for further control of the release of agent from the matrix. In particular embodiments, a first polymer forms the basal layer, and the bioactive agent is encased in a second polymer that is embedded in the matrix, the second polymer having a slower dissolution rate than the first polymer.

Any biocompatible dissolvable polymer may be used with compositions of the invention. Exemplary polymers include a water soluble polymer, a pH dependent polymer, a thermoplastic polymer, or a combination thereof. In addition to the polymer matrix, compositions of the invention may be formulated with other compounds that impart beneficial properties to the composition. Exemplary additional compounds include plasticizers, buffering agents, sweeteners, and coloring agents. In certain embodiments, the matrix is thicker than that used in orally-dissolving films (e.g., greater than 0.5 mm in thickness). Generally, the matrix is formed so that it has a thickness from about 0.5 mm to about 10 mm at its thickest point.

Any bioactive agent may be used with compositions of the invention. Particularly, useful agents are those that are not usually suitable for oral administration because they are poorly absorbed, even after they are dispersed in the stomach, due to low solubility and slow dissolution in the gastric fluids or modification of the active drug during liver metabolism. The target bioactive agent may be incorporated at a dose level, which can be determined by one of skill in the art, including dose levels higher than achievable using conventional orally-dissolvable films. In certain embodiments, the bioactive agent is incorporated at dose levels from about 15% to about 50% of the matrix by weight.

Compositions of the invention can be formulated to include a single bioactive agent or a plurality of bioactive agents. In certain embodiments, the matrix includes a plurality of bioactive agents, at least one of which is a B vitamin molecule or salt thereof. Exemplary types of B vitamin molecules include L-folinic acid or salts thereof, methylcobalamin or salts thereof, 5-deoxyadenosyl cobalamin or salts thereof, and cyanocobalamin or salts thereof. In other embodiments, the film includes a plurality of different B vitamin molecules and at least one additional bioactive agent. In particular embodiments, at least one of the bioactive agents is an agent or therapy which may deplete folate concentration.

Aspects of the invention also include a method for delivering a bioactive substance within an oral cavity of a mammal (e.g., a human) that involves administering a matrix that includes a polymer and one or more bioactive agents, in which the matrix is prepared so as to have a heterogeneous thickness.

### DETAILED DESCRIPTION

The invention generally relates to consumable matrices for delivering a bioactive agent to an oral cavity of a mammal. In certain aspects, the invention provides a consumable matrix that releases a bioactive agent within an oral cavity of a mammal, the matrix includes a polymer and one or more bioactive agents. The matrix is produced such that it has a heterogeneous thickness. Compositions of the invention provide for both controlled, rapid and sustained release of a bioactive agent from the matrix.

Any biocompatible polymer or combinations of multiple polymers may be used with compositions of the invention. In the embodiment of the invention, the matrix-forming polymer is a hydrocolloid, a water-soluble polymer matrix-forming material which is pharmaceutically acceptable. Exemplary hydrocolloids include polyanionic, polycationic or uncharged polymer material. These can include cellulose polymers (synthetic) such as hydroxyl propyl methyl cellulose (HPMC) hydroxyl propyl cellulose (HPC), methyl cellulose (MC), carboxymethylcellulose (CMC), starches and natural cellulose polymers such as acacia, tragacanth, carrageenan, pullulan and other water-soluble polymers including polystyrene sulphonates, polyethylene oxides, polyethylene glycols, polyacrylic acids, polybenzeinesulphonic acids, polyethylenamine hydrochloride, polyvinyl pyrrolidone (PVP), and gelatine. The water soluble polymeric materials can also be pectin and derivatives, guar gum, zanthan gum, gellan sodium salt, propyleneglycolog alginate starches, modified starches, hydroxyethyl starch, pullulan, carboxymethyl starch, gum gharti, okra gum, karaya gum, dextrans, dextrins and maltodextrins, konjac, acemannan from aloe, locust bean gum, tara gum, quince seed gum, fenugreek seed gum, sclerogucan, gum Arabic, psyllium seed gum, tamarind gum, oat gum, carrageenas, sckerglucan, succinoglucan, larch arabinogalactan, flaxseed gum, chondroitin sulphates, hyaluronic acid, curlan, chitosan, deacetylated jonjac and rhizobium gum.

Additionally, in embodiments of the invention, the water soluble hydrocolloid may be a polypeptide or protein exemplified by gelatins, albumins, milk proteins, soy protein and whey protein. The hydrocolloid may be further be selected from a group of synthetic hydrocolloids exemplified by any of the following polyethylene-imine, hydroxyethyl cellulose, sodium carboxymethyl cellulose, carboxymethyl cellulose, sodium carboxyethyl cellulose, hydroxyl propylmethyl cellulose, hydroxypropyl cellulose, methyl cellulose, ethyl cellulose, polyacrylic acids, low molecular weight polyacrylamides and their sodium salts, polyvinylpryyolidone, polyethylene glycols, polyethylene oxides, polyvinyl alcohols, pluoronics, tetronics and other block co-polymers, carboxyvinyl polymers and colloidal silicon dioxide.

In addition to hydrocolloids and the bioactive active agent, the matrix may contain any or all of the following ingredients: emulsifying agents, suspending agents, wetting agents, suspending agents, taste-modifying agents, solubilising agents, wetting agents, suspending agents, plasticizers, water soluble inert fillers, preservatives, buffering agents, effervescence agents, colouring agents and stabilizers. For enhanced absorption of the bioactive agent, it may also be advantageous to add a permitted permeation enhancer to the formulation. Examples of permeation enhancers include aprotinin, azone, benzylalkonium chloride, cetylpyridinium chloride, cyclodextrins, dextran sulphate, menthol, sodium glycodeoxycholate, sodium taurodeoxycholate and 23-lauryl ether.

Emulsifying agents may also be used to alter release properties, including polyvinyl alcohol, Sorbian esters, cyclodextrins, benzyl benzoate, glyceryl monstereate, polyxytheylene alkyl ethers, polyxyethylene stearates, poloxamer, plyoxythylene castor oil derivatives, hydrogenatated vegetable ouls, bile salts, polysorbates and ethanol.

Plasticizers including glycerine, sorbitol, propylene glycol, polyethelene glycol, triacetin, triethyl esters may provide rigidity and slow down release of the bioactive agent from the matrix.

Water soluble inert fillers include mannitol, xylitol, sucrose, lactose, malodextrin, dextran, dextrin, modified starches, dextrose, sorbitol, and dextrates may be used to modify the concentration and release properties of the bioactive agent from the matrix.

Effervescence and buffering agents include acidulants and alkalising agents including citric acid, fumaric acid, lactic acid, tartaric aid, malic acid, as well as sodium citrate, sodium bicarbonate and carbonate, sodium or potassium phosphate and magnesium oxide may be included in the matrix to assist stability and release of the pharmaceutical compound(s) from the matrix.

Flavoring agents include the essential oils or water soluble extracts of menthol, wintergreen peppermint, sweet mint, vanillin, cherry, chocolate, cinnamon, clove, lemon, orange, raspberry, rose, spice, violet, herbal, fruit, strawberry, grape, pineapple, peach, kiwi, papaya, mango, coconut, apple, coffee, plum, watermelon, nuts, durean, green tea, grapefruit, banana, butter, camomile, sugar, dextrose, lactose, talin, glycyrrhizin, sucralose, aspartame, saccharin, sodium saccharin, sodium cyclamate and honey, may be included for taste-masking.

Coloring agents may include F, D and C coloring agents, natural cooking agents and natural juice concentrates, pigments and pacifying agents such as titanium oxide, silicon dioxide and zinc oxide may be used to improve the appearance of the resultant matrix containing the bioactive agent.

Stabilizers and preservatives may include antioxidants, chelating agents and enzyme inhibitors such as acerbate acid, vitamin E, butylated hydroxyansiole (BHA), butylated hydoxytoluene (BHT), propyl gallate, dilauryl thiodpripionate, thiodipropianoicac acid, gum, guasiac, citric acid, edetic acid and its salts, glutathione, and microbial agents, sodium benzoate, parabens and derivatives, sorbic acid and its salts, propionic acid and its salts, sulphur dioxide and sulphites, acetic acid and acetates, nitrites and nitrates and others may be used to improve shelf life and stability of the matrix and bioactive agents.

An increase in saliva production may also be helpful in securing the rapid dissolution of the matrix formulation. This can normally be achieved by the addition of one or more sialagogues (saliva stimulating agents) to the formulation. Examples of such agents include organic acids such as citric acid, malic acid, ascorbic acid, tartaric acid and lactic acid.

In embodiments of the invention, the composition may contain a matrix-forming polymer(s) at a concentration in the range of 5-99% of the dry weight of the matrix, and typically greater than 5-10% of the dry weight of the matrix. The resulting sheets have ‘dry tack’ and ‘wet tack’ properties that improve ease of handling and use. The low ‘dry tack’ properties of the sheet provide for a physically attractive and easily handed material that is neither fragile nor sticky and can be easily removed from packaging and placed on the oral/mucosal surface. These properties render the matrices suitable for easy manufacture, packaging, handling and application.

Generally, polymer matrices are classified according to their porosity. In macroporous or microporous matrices, drug diffusion occurs essentially through pores, whereas non-porous systems have no pores and the molecules diffuse through the molecular networks of the polymers in the matrix. The polymer matrix of the invention can be prepared to be porous (e.g., macroporous or microporous) or non-porous with resultant impact on the release properties of the matrix.

Compositions of the invention include at least one bioactive agent, and in certain embodiments, more than one bioactive agent, such as two, three, four, or more bioactive agents. In some cases, two or more active pharmaceutical compounds may be included in the matrix with independently selected release profiles in order to optimise therapeutic efficacy for a chosen active drug compound. The bioactive agent chosen will depend on the condition or disease to be treated. Particularly, useful agents are those that are not usually suitable for oral administration because they are poorly absorbed, even after they are dispersed in the stomach, due to low solubility and slow dissolution in the gastric fluids or modification of the active drug. Other particularly useful agents are those that are administered to treat people that have neuropathies, neuromuscular disorders or poisoning by neuromuscular blocking agents, since such diseases generally prevent or interfere with a patient swallowing.

In certain embodiments, the bioactive agent is an aminopyridine, such as 4-aminopyridine, 2,4-diaminopyridine. 3,4-diaminopyridine and 3,4,5-triaminopyridine. Aminopyridines are a class of compounds that block potassium channels as exemplified by 4-aminopyridine (H₂NC₅H₄N), a central nervous stimulant that has recently been licensed for human therapeutic use as well as having a long history of veterinary use to reverse the effects of certain anesthetics and sedatives as well as being used as a pest bird flock deterrent. By blocking the transient efflux of potassium through voltage-gated potassium channels along the axon or at a nerve terminal or both, aminopyridines prolong the action potential and thus can improve signal conduction in damaged or dysfunctional nerves. Accordingly, aminopyridines are potentially valuable for treating diseases, disorders or conditions associated with impaired or diminished signal transmission in neurons. Aminopyridines are further exemplified in U.S. Pat. No. 4,775,536; U.S. Pat. No. 4,684,516; U.S. Pat. No. 4,265,874; U.S. Pat. No. 5,401,868; U.S. Pat. No. 5,684,018; U.S. Pat. No. 7,060,259; U.S. Pat. No. 7,244,748; U.S. Pat. No. 7,803,801; US 2011/0130429, WO 2005/046575; WO 2010/008886; Adler, et al. (Toxicon 34(2):237-249 (1996)); Alexander, et al. (J. Med. Chem. 31:318-322 (1988)); Amsberry (Pharmaceutical Research 8(4):455-461 (1991)); Bradshaw and Westwell (Current Medicinal Chemistry 11:1241-1253 (2004)); and Gershonov, et al. (J. Med. Chem. 43(13):2530-2537 (2000)), the content of each of which is incorporated by reference herein in its entirety.

In certain embodiments, the bioactive agent is a sodium channel blocker. Voltage-gated sodium channels are complex membrane proteins that are widely expressed in neuronal, neuroendocrine, skeletal muscle and cardiac cells. These channels activate in response to membrane depolarization and, in most electrically excitable tissues, these channels are responsible for the rapid influx of sodium ions during a rising phase of an action potential.

A blocker of such sodium channels refers to a compound that impairs conduction of sodium ions through sodium channels. Under pathological conditions (such as ischaemia) sodium channels become abnormally activated resulting in an excessive flow of sodium ions into the cytoplasm. The rise in cellular sodium ions then causes a large inflow of calcium ions leading to the activation of several mechanisms that lead to irreversible loss of function and subsequent degeneration. It is possible to produce pharmacological agents capable of stopping excessive activity of sodium channels without adversely affecting their normal function. Indeed, this is the principal mode of action of several widely-used and well-tolerated antiepileptic drugs (e.g. phenytoin, carbamazapine and lamotrigine).

Sodium channel inhibitors have been shown to be protective towards neurons in the grey matter in several models of cerebral ischaemia. More recently, it has become evident that certain sodium channel blockers are highly effective in protecting axons in the in vitro optic nerve from irreversible damage imposed by severe deprivation of oxygen and glucose and in experimental models of spinal cord injury. (Stirling and Stys, Trends Mol Med. 16:160-70, 2010). Blockers of voltage-gated sodium channels have proven therapeutic value in local anaesthesia, cardiac arrythmia, pain, bipolar disorder and epilepsy and are currently under investigation for stroke and other disorders. See e.g., Clare et al. (Drug Discov Today. 5: 506-520, 2000).

Sodium channel blockers that have been used to treat neurological and neurodegenerative disorders are shown in Smith et al. (WO 99/52522), Harling et al. (WO 98/46574), Bountra et al. (WO 00/61231), Harbige et al. (U.S. 20040229873), and Kapoor et al. (The Lancet Neurology 9:681-688, 2010), the content of each of which is incorporated by reference herein in its entirety. Exemplary disorders include epilepsy, multiple sclerosis, Alzheimer's disease, fronto-temporal dementia, Lewy body dementia, Parkinson's disease, Huntington's disease, and motor neuron disease. Other neurological and neurodegenerative disorders that are treated with sodium channel blockers are shown in The Merck Manual of Diagnosis and Therapy, published by Merck Sharp & Dohme Corp., Whitehouse Station, N.J., U.S.A., 2004-2010, the content of which is incorporated by reference herein in its entirety.

Without being limited by any particular theory or mechanism of action, it is believed that in neurological and neurodegenerative disorders, axons succumb to damage through the loss of ionic homeostasis through sodium/potassium ATPase failure, sodium entry through persistent sodium channels and potassium efflux, causing the reversal of the sodium/calcium exchanger and a resultant accumulation of intra-axonal calcium. Many factors probably play a role in this process, including nitric oxide (NO). It has previously been shown that axons exposed to NO in vivo can undergo degeneration, especially if the axons are electrically active during NO exposure (Kapoor et al., The Lancet Neurology 9:681-688, 2010; and Stirling and Stys, Trends Mol Med. 16:160-70, 2010). The axons may degenerate because NO can inhibit mitochondrial respiration, leading to intraaxonal accumulation of Na⁺ and Ca²⁺ ions. It has been found that axons can be protected from NO-mediated damage using Na⁺ channel blockers (Kapoor et al., The Lancet Neurology 9:681-688, 2010).

Sodium channel blockers that have been used to treat psychiatric disorders are shown in Stahl (J Clin Psychiatry, 65(6):738-739, 2004). Exemplary disorders include anxiety, bipolar disorder, mood disorders and schizophrenia. Other psychiatric disorders that are treated with sodium channel blockers are shown in The Merck Manual of Diagnosis and Therapy, published by Merck Sharp & Dohme Corp., Whitehouse Station, N.J., U.S.A., 2004-2010, the content of which is incorporated by reference herein in its entirety.

Sodium channel blockers that have been used to treat disorders involving pain are shown in Bhattacharya (Neurotherapeutics, 6(4):663-678, 2009). Exemplary disorders include migraine, neuropathic pain, and chronic pain. Other disorders involving pain that are treated with sodium channel blockers are shown in The Merck Manual of Diagnosis and Therapy, published by Merck Sharp & Dohme Corp., Whitehouse Station, N.J., U.S.A., 2004-2010, the content of which is incorporated by reference herein in its entirety.

Sodium channel blockers have also been used to treat stroke (Hewitt et al., Brain Res., 898(2):281-287, 2001), glaucoma (Hains et al., Invest Ophthalmol Vis Sci., 46(11):4164-4169, 2005), uveitis (U.S. Pat. No. 6,221,887), traumatic brain and spinal cord injury (Schwartz et al., J Neurosurg., 94(2 Suppl):245-256, 2001), and cerebral ischemia (Hewitt et al., Brain Res., 898(2):281-287, 2001). The content of each of these references is incorporated by reference herein in its entirety.

In certain embodiments, the sodium channel blocker is a compound of the lamotrigine family of compounds. Such compounds include analogs, derivatives, salts, and prodrugs of lamotrigine. Exemplary compounds of the lamotrigine family are shown in Harbige et al. (U.S. 20040229873), the content of which is incorporated by reference herein in its entirety. Exemplary compounds include lamotrigine; 3,5-diamino-6-(2,3-dichlorophenyl)-1,2,4-triaz-ine; Sipatrigine; 4-amino-2-(4-methyl-1-piperazinyl)-5-(2,3,5-trichlorophe-nyl)-pyrimidine; 2,4-diamino-5-(2,3-dichlorophenyl)-6-(fluoromethylpyrimid-ine); R-(−)-2,4-diamino-6-fluoromethyl-5-(2,3,5-trichlorophenyl)-pyrimidin-e; 4-amino-2-(1-piperazinyl)-5-(2,3,5-trichlorophenyl)-pyrimidine (active Sipatrigine metabolite); 4-amino-2-(4-methyl-1-piperazinyl)-5-(2,3,5-trichlorophenyl)-6-trifluoromethylpyrimidine; 2,4-diamino-5-(2,3,5-trichloroph-enyl)-trifluoromethylpyrimidine; 2,4-diamino-5-(2,3,5-trichlorophenyl)-6-m-ethoxymethylpyrimidine; 4-amino-6-methyl-2-(4-methyl-1-piperazinyl)-5-(2,3-,5-trichlorophenyl)-pyrimidine; 4-amino-2-(4-propyl-1-piperazinyl)-5-(2,3,-5-trichlorophenyl)-pyrimidine; and 2,4-diamino-5-(2,3,5-trichlorophenyl)-py-rimidine. Several of these compounds are described in U.S. Pat. Nos. 5,635,507, 5,597,828, 5,684,005, 5,587,380, 5,712,276 and 5,712,277 all of which are incorporated herein by reference.

In certain embodiments, the sodium channel blocker is lamotrigine, carbamazepine, oxcarbazepine, valproic acid, sipatrigine, 4030w92, 202w92, 78c90 (active sipatrigine metabolite), 440c89, 149C89, 722c90, 279c90 or 1003c87. In particular embodiments, the sodium channel blocker is lamotrigine. Lamotrigine is commercially available from GlaxoSmithKline under the product name LAMICTAL, and is described in Bountra et al. (WO 00/61231).

In certain embodiments, the bioactive agent is a saponin. Saponins are a class of chemical compounds that include one or more hydrophilic glycoside moieties combined with a lipophilictriterpene derivative. Saponins are often large molecules that display poor, absorption into the bloodstream following oral administration. Exemplary saponins are shown for example in PCT/GB04/05398; U.S. Pat. No. 7,906,493; PCT/GB06/02500; US 2012/0142620; PCT/GB06/02518; U.S. Pat. No. 7,998,943; PCT/GB06/02301; and US 2007/0010460, the content of each of which is incorporated by reference herein in its entirety.

In certain embodiments, the matrix includes a plurality of bioactive agents, at least one of which is a B vitamin molecule or salt thereof. Exemplary types of B vitamin molecules include L-folinic acid or salts thereof, methylcobalamin or salts thereof, 5-deoxyadenosyl cobalamin or salts thereof, and cyanocobalamin or salts thereof. In other embodiments, the film includes a plurality of different B vitamin molecules and at least one additional bioactive agent. In particular embodiments, the at least one of the bioactive agents is an agent that depletes folate concentration. In certain embodiments, the film includes a B vitamin molecule and a sodium channel blocker.

A “B vitamin molecule”, as used herein, refers to any or all of a complex of several vitamins that were discovered during early studies of human nutrition, exemplified by vitamin B1 (thiamine), vitamin B2 (riboflavin), vitamin B3 (vitamin P or vitamin PP, or niacin), vitamin B5 (pantothenic acid), vitamin B6 (pyridoxine and pyridoxamine), vitamin B7 (vitamin H, vitamin B-w, or biotin), vitamin B9 (vitamin M, vitamin B-c, or folic acid), and vitamin B12 (cyanocobalamin).

A B vitamin molecule also includes without limitation, “nonhuman forms” discovered by study of nutrition in other life form (animals, bacteria, yeast, etc.) such as vitamin B4 (adenine), vitamin B8 (ergadenylic acid), vitamin B10 (para-aminobenzoic acid), vitamin B11 (salicylic acid or vitamin S), vitamin B13 (pyrimidinecarboxylic acid or orotic acid), vitamin B14 (a mixture of vitamin B10 and vitamin B11), vitamin B15 (pangamic acid or dimethylglycine), vitamin B16, vitamin B17 (amygdalin), vitamin B22, vitamin B-t (L-carnitine), and vitamin B-x (para-aminobenzoic acid).

The B vitamins often work together to deliver a number of health benefits to the body, such as, bolstering metabolism, maintaining healthy skin and muscle tone, enhancing immune and nervous system function, and promoting cell growth and division. Combined, the B vitamins assist in combating the symptoms and causes of stress, depression, and cardiovascular disease. B vitamins are water soluble and are dispersed throughout the body and must be replenished daily, as any excess is excreted generally in the urine.

A “vitamin B2 molecule”, as used herein, refers to any or all of vitamin B2, riboflavin or vitamin G. As used herein, this term includes also the coenzyme forms, flavin adenine dinucleotide (FAD) and flavin adenine mononucleotide (FMN). B2 molecules are easily absorbed, water-soluble micronutrients that support energy production by aiding in the metabolism of fats, carbohydrates, and proteins. Vitamin B2 molecules are also needed for red blood cell formation and respiration, antibody production, and for regulating human growth and reproduction. They function as antioxidants by scavenging damaging particles in the body known as free radicals. Vitamin B2 molecules are important for healthy skin, nails, hair growth and general good health, including regulating thyroid activity.

Vitamin B2 deficiency manifests itself as cracks and sores at the corners of the mouth, eye disorders, inflammation of the mouth and tongue, skin lesions, dermatitis, dizziness, hair loss, insomnia, light sensitivity, poor digestion, retarded growth, and the sensation of burning feet.

An exemplary structure of the vitamin B2 molecule is shown below:

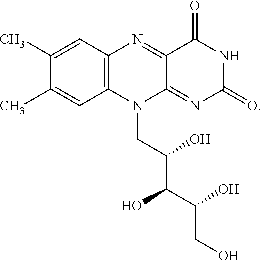 [SMILES: Cc1cc2nc3c(=O)[nH]c(=O)nc-3n(C[C@H](O)[C@H](O)[C@H](O)CO)c2cc1C]

A “vitamin B3 molecule”, as used herein, refers to any or all of vitamin B3, niacin, or nicotinic acid. These include the amide form, nicotinamide or niacinamide. Vitamin B3 molecules are water-soluble vitamins whose derivatives such as NADH, NAD, NAD⁺, and NADP play important roles in energy metabolism in the living cell and DNA repair. These molecules also assist the body make various sex and stress-related hormones in the adrenal glands and other parts of the body. A vitamin B3 molecule is effective in improving circulation and reducing cholesterol levels in the blood.

Lack of a vitamin B3 molecule causes the deficiency disease pellagra. A mild B3 deficiency causes a slowdown of the metabolism, which in turn causes a decrease in cold tolerance and is a potential contributing factor towards obesity.

In vivo synthesis of a vitamin B3 molecule is initiated from the 5-membered aromatic heterocycle of the amino acid tryptophan, which is cleaved and rearranged with the alpha amino group of tryptophan into the 6-membered aromatic heterocycle of a vitamin B3 molecule. The reaction proceeds as follows: tryptophan->kynurenine->3-hydroxy kynurenine (B6 enzyme needed)->vitamin B3 molecule. The liver can synthesize vitamin B3 molecules from the amino acid tryptophan, and the synthesis is slow and requires vitamin B6, i.e., 60 mg of tryptophan are required to make one milligram of a vitamin B3 molecule.

An exemplary structure of the vitamin B3 molecule is shown below:

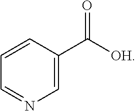

A “vitamin B6 molecule”, as used herein, refers to any or all of vitamin B6, pyridoxine, pyridoxal, and pyridoxamine. These molecules are converted to pyridoxal 5′-phosphate (PLP) in the liver. PLP is an important cofactor for numerous metabolic enzymes, such as aminotransferases, amino acid racemases, and amino acid decarboxylases, most of which have amino group-containing compounds as substrates. In the absence of PLP, a substantial number of cellular biosynthetic and catabolic pathways would cease to function.

Two pathways of de novo PLP synthesis are known, the PdxA/PdxJ pathway and the PDX1/PDX2 pathway. Organisms appear to contain either one or the other pathway of de novo PLP synthesis. Vitamin B6 comprises, in addition to PLP, precursors of PLP in phosphorylated and non-phosphorylated forms, and these compounds are referred to as B6 vitamers. Non-phosphorylated vitamers pyridoxine, pyridoxal and pyridoxamine can be taken up by many bacteria, fungi, plants, and mammalian cells and converted into PLP by a salvage pathway.

An exemplary structure of the vitamin B6 molecule is shown below:

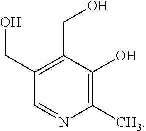 [SMILES: Cc1ncc(CO)c(CO)c1O]

A “vitamin B9 molecule”, as used herein, refers to any or all vitamin B9, folic acid and folate. The B9 molecule is a water-soluble vitamin that is important for the production and maintenance of new cells, particularly during periods of rapid cell division and growth such as infancy and pregnancy. The B9 molecule is needed to replicate DNA and synthesize RNA, and is involved in the synthesis, repair, and functioning of DNA. A deficiency of folate may result in damage to DNA that may lead to cancer. Both adults and children need vitamin B9 molecules to make normal red blood cells and prevent anemia.

Signs of vitamin B9 deficiency include diarrhea, loss of appetite, weight loss, weakness, sore tongue, headaches, heart palpitations, irritability, and behavioral disorders. In adults, anemia is a sign of advanced vitamin B9 deficiency. In infants and children, vitamin B9 deficiency can slow growth rate.

An exemplary structure the vitamin B9 molecule is shown below:

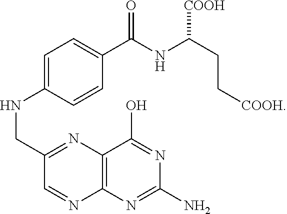 [SMILES: Nc1nc(O)c2nc(CNc3ccc(C(=O)N[C@@H](CCC(=O)O)C(=O)O)cc3)cnc2n1]

A “vitamin B12 molecule”, as used herein, refers to any or all of a group of cobalt containing tetrapyrrole compounds known as corrinoids. Examples include, cobalamin, cyanocobalamin, hydroxocobalamin, and thiocyanate cobalamin. The structure of vitamin B12 molecules comprises a nucleotide (base, ribose and phosphate) attached to a corrin ring which is made up of four pyrrole groups and an atom of cobalt in the center. The cobalt atom bonds to a methyl group, a deoxyadenosyl group, and a hydroxyl group or a cyano group. A vitamin B12 molecule includes the coenzyme forms of vitamin B12, i.e., methylcobalamin and 5-deoxyadenosylcobalamin(adenosylcobalamin).

Deficiency of vitamin B12 results in hematological, neurological and gastrointestinal effects. The hematological effects are caused by interference with DNA synthesis. The hematologic symptoms and signs of vitamin B12 deficiency, include hypersegmentation of polymorphonuclear leukocytes, macrocytic, hyperchromic erythrocytes, elevated mean corpuscular volume (MCV), elevated mean corpuscular hemoglobin concentration (MCH, MCHC), a decreased red blood cell count, pallor of the skin, decreased energy and easy fatigability, shortness of breath and palpitations.

The neurological effects of the vitamin B12 deficiency include tingling and numbness in the extremities (particularly the lower extremities), loss of vibratory and position sensation, abnormalities of gait, spasticity, Babinski's responses, irritability, depression and cognitive changes (loss of concentration, memory loss, dementia). Visual disturbances, impaired bladder and bowel control, insomnia and impotence may also occur.

Gastrointestinal effects of vitamin B12 deficiency include intermittent diarrhea and constipation, abdominal pain, flatulence and burning of the tongue (glossitis). Anorexia and weight loss are general symptoms of vitamin B12 deficiency.

Pathologies or defects can reduce efficiency or function of this pathway, such as an autoimmune condition involving formation of antibodies against the cells producing intrinsic factor; presence of a fish tapeworm; or the after-effects of surgery to the small intestine which results in the surface of the small intestine being insufficient to obtain B12 and intrinsic factor. These pathologies or defects result in less efficient absorption of vitamin B12, and could be ameliorated by administration of a higher dosage of vitamin B12.

An exemplary structure of a vitamin B12 molecule is shown below:

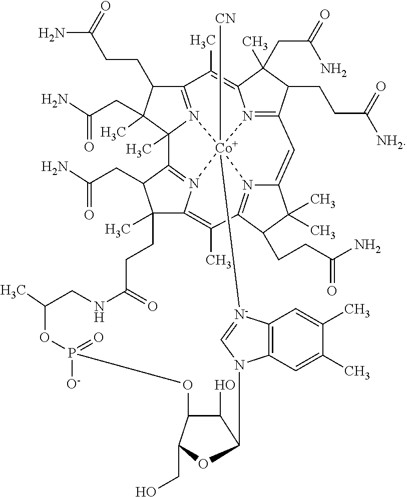

In certain embodiments, the B vitamin molecule is a vitamin B9 molecule, which includes, salts, analogs, derivatives, or Pro-Drugs thereof. In particular embodiments, the B vitamin is a derivative of a vitamin B9 molecule. Exemplary derivatives of vitamin B9 molecules include tetrahydrofolate, methyl-tetrahydrofolate (CH₂-THF), and 10-formyl-tetrahydrofolate (10-CHO-THF).

In the form of a series of tetrahydrofolate compounds, folate derivatives are coenzymes in a number of single carbon transfer reactions biochemically, and also is involved in the synthesis of dTMP (2′-deoxythymidine-5′-phosphate) from dUMP (2′-deoxyuridine-5′-phosphate).

The pathway in the formation of tetrahydrofolate (Fe) is the reduction of folate (F) to dihydrofolate (FH₂) by folate reductase, and then the subsequent reduction of dihydrofolate to tetrahydrofolate (FH₄) by dihydrofolate reductase. Methylene tetrahydrofolate (CH₂FH₄) is formed from tetrahydrofolate by the addition of methylene groups from one of three carbon donors: formaldehyde, serine, or glycine. Methyl tetrahydrofolate (CH₃—FH4) can be made from methylene tetrahydrofolate by reduction of the methylene group, and formyl tetrahydrofolate (CHO—FH₄, folinic acid) is made by oxidation of the methylene tetrahydrofolate.

THF is the folate product of the methionine synthase reaction, in addition to the reduction of folic acid. 5-Methyltetrahydrofolate is generated by conversion of 5,10-methylenetetrahydrofolate into 5-methyltetrahydrofolate via the enzyme methyleneterahydrofolate reductase (MTHFR). 5,10-Methylenetetrahydrofolate is regenerated from tetrahydrofolate via the enzyme serine hydroxymethyltransferase. 5-Methyltetrahydrofolate donates a methyl group to homocysteine, in conversion of homocysteine to L-methionine. The enzyme that catalyzes the reaction is methionine synthase. Vitamin B₁₂ is a cofactor in the reaction. This reaction, in which vitamin B₁₂ is a cofactor, is of great importance in the regulation of serum homocysteine concentration. The L-methionine produced in the reaction can participate in protein synthesis and is also a major source for the synthesis of S-adenosyl-L-methionine (SAMe). The methyl group donated by 5-methyltetrahydrofolate to homocysteine in the formation of L-methionine is used by SAMe in a number of transmethylation reactions involving nucleic acids, phospholipids and proteins, as well as for the synthesis of epinephrine, melatonin, creatine and other molecules. Tetrahydrofolate is the folate product of the methionine synthase reaction. 5-0-Methylenetetrahydrofolate, in addition to its role in the metabolism of homocysteine, supplies the one-carbon group for the methylation of deoxyuridylic acid to form the DNA precursor thymidylic acid. This reaction is catalyzed by thymidylate synthase and the folate product of the reaction is dihydrofolate. Dihydrofolate is converted to tetrahydrofolate via the enzyme DHFR.

Folinic acid is a 5-formyl derivative of THF that is readily converted to tetrahydrofolate and thus has vitamin activity which is equivalent to folic acid. Leucovorin is a commercially available agent that is a mixture of diastereoeisomers of folinic acid. The biologically active compound of the mixture is the (−)-1-isomer, known as citrovorum factor or (−)-folinic acid. Leucovorin does not require reduction by the enzyme DHFR in order to participate in reactions utilizing folates, principally one-carbon transfer reactions. Administration of leucovorin can counteract the therapeutic and toxic effects of folic acid antagonists such as methotrexate, which act by inhibiting dihydrofolate reductase. Folinic acid is available as a calcium salt for parenteral or oral administration.

Leucovorin is used as an antidote to drugs which act as DHFR inhibitors. Leucovorin is employed in injection form as an aqueous bacteriostatic preparation containing leucovorin present as the calcium salt pentahydrate of N-[4-[[(2-amino-5-formyl-1,4,5,6,7,8-hexahydro-4-oxo-6-pteri-dinyl)-methyl-]amino-]benzoyl]-L-glutamic acid. Each 5 mg of leucovorin is equivalent to 5.4 mg of anhydrous leucovorin calcium or 6.35 mg of leucovorin calcium pentahydrate.

In other embodiments, the B vitamin molecule is a Pro-Drug of tetrahydrofolate. Methods of making prodrugs are described above. Exemplary Pro-Drugs of tetrahydrofolate include the general structure of tetrahydrofolate having certain Pro-Drug moieties attached at the 5-position include the following: amides, including natural and unnatural amino acid amides; carbamates; aryl phosphonic acids; and aryl esters. An exemplary Pro-Dug of tetrahydrofolate is shown as Formula I below:

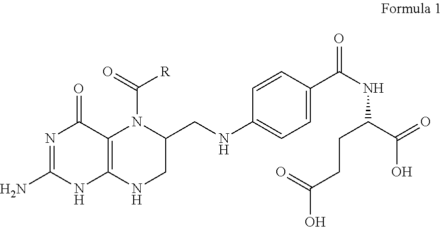

wherein R is selected from the group consisting of: an amino acid attached through the carboxyl group of the amino acid; an alkyl; an aryl; an alkoxy; an aryloxy; an aryl phosphonic acid; and an aryl ester comprising either of the below side chains:

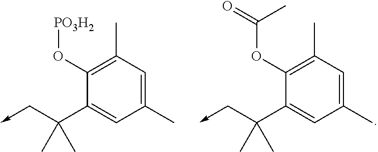

Generally, the polymers include either water soluble polymers, pH-dependent polymers or thermoplastic polymers or combinations thereof along with suitable amounts of water, water soluble/miscible/water insoluble plasticizers, buffering agents, sweeteners, coloring agents and drugs. The matrix will usually be prepared in solution, suspension or emulsion and then subsequently dried into defined physical proportions. Loading and dispersion of the bioactive agent in the polymer matrix is effected by blending the materials in a suitable solvent leading to gradual and uniform dissolution of the bioactive agent throughout the matrix. Bioactive agents can also be loaded using supercritical fluid techniques since most agents often exhibit good solubility in super critical fluids.

In a preferred embodiment, the percentage dry weight concentration of at least single ingredients incorporated in a matrix in each of the following categories is as follows: emulsifying agent (0.1-10%), plasticizer (0.5-20%), active agents (0.01-80%), taste-modifying agents (0.1-10%), colouring agents (0.01-5%), water insoluble inert fillers (0.5-50%), preservatives (0.01-10%), buffering agents (0.1-10%) and stabilizers (0.01-5%).

Any method known in the art may be used to produce films of the invention. Exemplary methods include solvent casting, semisolid casting, hot melt extrusion, solid dispersion extrusion, and rolling. These methods are known in the art and described, for example in Arya et al. (Int. J. CehmTech, 2(1):576-583, 2010), the content of which is incorporated by reference herein in its entirety.

In the solvent casting method, water soluble polymers are dissolved in water and the bioactive agent along with other excipients are dissolved in a suitable solvent. Then, both solutions are mixed and stirred and finally cast onto a flat substrate and dried.

In the semisolid casting method, a solution of water-soluble film forming polymer is prepared. The resulting solution is added to a solution of acid insoluble polymer (e.g. cellulose acetate phthalate, cellulose acetate butyrate), which is prepared in ammonium or sodium hydroxide. Then, an appropriate amount of plasticizer is added so that a gel mass is obtained. Finally the gel mass is cast into films or ribbons using heat controlled drums. The ratio of the acid insoluble polymer to matrix forming polymer should be about 1:4 in such cases.

In the hot melt extrusion method, the bioactive agent is mixed with carriers in solid form. The solid form is placed in the extruder, which includes heaters, allowing the solid form to be melted in the extruder. The moltern mixture is then shaped by the extruder.

In the solid dispersion extrusion method, immiscible components are co-extruded with bioactive agent and then solid dispersions are prepared. Finally the solid dispersions are shaped means of dies.

In the rolling method, a solution or suspension containing a bioactive is rolled on a carrier. The solvent is mainly water or a mixture of water and alcohol. The film is dried on the rollers and cut into desired shapes and sizes.

Generally, upon contact with release fluids (water or physiological media), the polymer in the matrix swells and drug dissolution occurs. Drug release kinetics may be affected by many factors such as polymer swelling, polymer erosion, drug dissolution/diffusion characteristics, drug distribution inside the matrix, drug/polymer ratio, system geometry and surface: volume ratio. These properties may be modified by a variety of means including but not limited to passing an inert gas into the matrix during production, resulting in a product with a honeycombed structure, which dissolves rapidly.

In certain embodiments, the surface area of the composition is modified to increase the surface area, e.g., the volume ratio. This can be accomplished by providing areas of reduced thickness in the composition. Compositions can be formulated as described above to have areas of a thickness of about 0.05 mm and other areas of a thickness of 1 mm. Any thickness ratios may be produced depending on the drug release rate that is desired. Other exemplary compositions have areas of 1 mm thickness and 3 mm thickness, areas of 2 mm thickness and areas of 5 mm thickness, areas of 1 mm thickness and areas of 10 mm thickness, etc. In certain embodiments, all of the areas of reduced thickness have the same thickness. For example, in a composition having 20 defined areas of reduced thickness, each of the 20 areas has a thickness of 1 mm while the thicker portion of the composition has a thickness of 5 mm. In other embodiments, at least two of the areas of reduced thickness have different thicknesses. For example, in a composition having 20 areas of reduced thickness, 10 of the areas have a thickness of 0.05 mm, and 10 of the areas have a thickness of 2 mm, while the thicker portion of the composition has a thickness of 7 mm.

In certain embodiments, the areas of reduced thickness are perforations in the matrix. The perforations can be made in the body of the matrix by several known methods such as use of pins/punches or laser drills or mechanical drills or conventional methods. One method of manufacturing a matrix that has perforations includes incorporating particles of a defined size into the polymer matrix during matrix preparation. After the matrix has been dried, the particles are removed from the matrix, e.g., through washing with a solvent in which the bioactive agent is not soluble, freezing or enzymatic removal of protein particles. Another method of producing a matrix including perforations includes drying the polymer matrix over a surface with a defined number of projections that reach through the matrix, and from which upon drying, the matrix sheet can be removed leaving a series of defined perforations in the matrix sheet.

The type of polymer chosen affects the dissolution rate of the matrix, and thus also affects the rate of release of the agent from the matrix. In certain embodiments, the matrix is made of a single polymer. In other embodiments, the polymer matrix includes a plurality of different polymers, optionally having different dissolution rates. Generally, when using multiple polymers, the different polymers are arranged as different layers of the matrix, which allows for further control of the release of agent from the matrix. Any of the above described methods may be used to generate multilayer matrices. In particular embodiments, a first polymer forms a matrix, and the bioactive agent is encased in a second polymer that is embedded in the matrix, the second polymer having a slower dissolution rate than the first polymer.

In some cases, it is desirable for release of the active agent to occur after dissolution of the matrix. For these applications, the active agent may be encapsulated in a material with the dissolution properties that are different from those of the main matrix. Encapsulation may also be utilised to achieve off-taste masking for active agents.

Any method known in the art may be used to test the properties of the produced matrices, and certain testing methods are described for example in Arya et al. (Int. J. CehmTech, 2(1):576-583, 2010), the content of which is incorporated by reference herein in its entirety. In certain embodiments, a matrix is produced having a dry thickness in the range of 1-25 mm, tensile strength greater than 2,500 psi, a modulus in the range of 25,000-350,000 psi, a disintegration time in the range of 1-300 seconds, a dissolution time in the range of 10-600 seconds and a percentage elongation less than 10%.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A consumable product that releases a bioactive agent within an oral cavity of a mammal, the product comprising a polymer matrix and one or more bioactive agents, wherein the matrix comprises a heterogeneous thickness.

2. The product according to claim 1, wherein the matrix is shaped as a sheet comprising one or more areas of reduced thickness.

3. The product according to claim 2, wherein the areas of reduced thickness are perforations in the sheet.

4. The product according to claim 1, wherein the matrix comprises a single type of polymer.

5. The product according to claim 1, wherein the matrix comprises a plurality of different polymers.

6. The product according to claim 5, wherein the different polymers are arranged as different layers of the matrix.

7. The product according to claim 5, wherein a first polymer forms a film, and the bioactive agent is encased in a second polymer that is embedded in the film, the second polymer having a slower dissolution rate than the first polymer.

8. The product according to claim 1, wherein the polymer is selected from the group consisting of: a water soluble polymer, a pH dependent polymer, a thermoplastic polymer, and a combination thereof.

9. The product according to claim 1, further comprising: a plasticizer, a buffering agent, a sweetener, a coloring agent, and a combination thereof.

10. The product according to claim 1, wherein the thickest portion of the matrix comprises a thickness from about 0.5 mm to about 10 mm.

11. The product according to claim 1, wherein the bioactive agent is at dosage levels from about 15% to about 50% of the matrix weight.

12. The product according to claim 1, wherein the product comprises a plurality of bioactive agents, at least one of which is a B vitamin molecule or salt thereof.

13. The product according to claim 12, wherein the B vitamin molecule is L-folinic acid or salts thereof.

14. The product according to claim 12, wherein the B vitamin molecule is methylcobalamin or salts thereof.

15. The product according to claim 12, wherein the B vitamin molecule is 5-deoxyadenosyl cobalamin or salts thereof.

16. The product according to claim 12, wherein the B vitamin molecule is cyanocobalamin or salts thereof.

17. The product according to claim 12, wherein the product comprises a plurality of different B vitamin molecules and at one additional bioactive agent.

18. The product according to claim 12, wherein at least one of the bioactive agents is an agent that depletes folate concentration.

19. A method for delivering a bioactive substance within an oral cavity of a mammal, the method comprising administering the consumable product of claim 1 to the oral cavity of the mammal.

20. The method according to claim 18, wherein the mammal is a human.
